(19) United States
(12) Patent Application Publication
(10) Pub. No.: US 2025/0352055 A1
(43) Pub. Date: Nov. 20, 2025

(54) SYSTEMS FOR EYE TREATMENT

(71) Applicants: Brenda PADILLA, Gilroy, CA (US); Sight Sciences, Inc., Menlo Park, CA (US)

(72) Inventors: Sam PARK, Menlo Park, CA (US); Scott HARSHMAN, Woodinville, WA (US); Robert DE SANTIAGO, Menlo Park, CA (US); Daniel O’KEEFFE, San Francisco, CA (US)

(73) Assignee: Sight Sciences, Inc., Menlo Park, CA (US)

(21) Appl. No.: 18/667,697

(22) Filed: May 17, 2024

(51) Int. Cl. A61B 3/10; A61F 7/00; A61F 9/00

(52) U.S. Cl. CPC A61B 3/10; A61F 9/0017; A61F 2007/0004

(57) ABSTRACT

Systems for eye treatment are disclosed where the system generally includes a housing defining a receiving channel and a first flexible support extending from the housing and having a first heating strip at a distal end of the first flexible support, wherein a proximal portion of the first flexible support defines a curved or bent portion which orients the first heating strip to extend in a first direction. The system also includes a second flexible support extending from the housing and having a second heating strip at a distal end of the second flexible support, wherein a proximal portion of the second flexible support defines a curved or bent portion which orients the second heating strip in a second direction. The flexible supports may further define a distal curved or bent portion proximal to the heating strips such that the heating strips are positioned to extend away from one another.

### FIELD OF THE INVENTION

The present invention relates to methods and apparatus for treatment of eye-related conditions such as dry eye syndrome and other related conditions. More particularly, the present invention relates to methods and apparatus for treating eye-related conditions with and tracking the condition of the eyes over time.

### BACKGROUND OF THE INVENTION

Tears are a complex mixture of water, lipids, mucus, proteins and electrolytes and this mixture helps to maintain a smooth, lubricious, and optically clear optical surface and also helps to protect the eyes from infection. The tear film has three basic layers: oil, water, and mucus and problems or disturbances in any of these layers can cause ocular surface problems including dry eye symptoms.

The outermost layer of the tear film is typically comprised of an oil layer containing fatty acids and lipids (meibum), which are produced primarily by sebaceous glands called the meibomian glands located along the eyelid margin. The oil layer smoothes the tear surface and retards evaporation of the aqueous or watery middle layer. However, if the meibomian glands fail to produce enough oil, produce suboptimal fatty acid mixtures, or if the glands become obstructed or clogged, the watery layer typically evaporates too quickly causing dry eyes. A blockage or inflammation of the meibomian glands can, among many things, lead to enlarged glands or infections, inspissated secretions, styes, chalazia, hordeolum, or preseptal cellulitis. Dry eyes are thus common in people whose meibomian glands are obstructed or functioning improperly. The aforementioned are some examples of meibomian gland dysfunction which is also sometimes referred to as evaporative dry eye.

The middle watery layer of tears is composed primarily of an aqueous solution, which is produced by the lacrimal glands and accessory glands (tear glands). The middle layer cleanses the eyes and washes away foreign particles or irritants, maintains a clear optical medium, and keeps the ocular surface moist. The innermost layer of the tear film is composed primarily of mucus, which helps to spread the tears evenly over the surface of the eyes. A lack of mucus in the tear film is also associated with dry eye syndrome (DES).

As discussed above, the meibomian glands are oil-secreting glands located within both the upper and lower eyelids. There are approximately 30 to 40 glands along the upper eyelid and approximately 20 to 30 glands along the lower eyelid with the ducts for each of the glands opening along the inner edge of the free margin of the respective lids by minute foramina through which their secretion is released to prevent the lids adhering to each other or to the ocular surfaces. An example of the location of the meibomian glands is illustrated in the cross-sectional view of the upper eyelid UL shown in FIG. 1A which illustrates the relative positioning of a single meibomian gland MG. Other glands and anatomical features are illustrated for reference, e.g., the glands of Wolfring GW, tarsus TR, gland of Moll GM, gland of Zeis GZ, gland of Krause GK, upper fornix UF, conjunctiva CN and cornea CR of the eye which is partially covered by the upper eyelid UL. As illustrated, the meibomian gland MG is positioned along a length of the upper eyelid UL (and lower eyelid LL) with the duct opening along the inner edge of the eyelid UL in proximity to a surface of the underlying eye.

FIG. 1B illustrates a front view of a patient's eye having the upper eyelid UL and lower eyelid LL in a closed position, such as when the patient blinks. As shown, the meibomian glands MG may be seen aligned adjacent to one another over both the upper UL and lower eyelids LL. FIG. 1C also shows a perspective view of a patient's eye in the open position to illustrate how the meibomian glands are typically aligned relative to one another when the patient's eye is opened.

Blinking is thought to be the primary mechanism to open the orifice of the meibomian glands to allow for the release of oil secretions from the glands. The natural blinking motion and blinking force causes the upper lid to pull or drag a sheet of the lipids secreted by the meibomian glands over the two underlying layers of the tear film thus forming the protective coating which limits the rate at which the underlying layers evaporate. It is estimated that at least 65% of meibomian gland disease or dry eye results from a defective lipid layer or an insufficient quantity of such lipids that results in accelerated evaporation of the aqueous layer. Hence, eyelid closure or blinking disorders, or other disorders that affect proper tear distribution, may also cause or exacerbate meibomian gland dysfunction or dry eye.

As the eyelids close in a total blink, the superior and inferior fornices, which hold a reservoir of tears, are compressed by the force of the preseptal muscles and the eyelids move toward one another. The upper eyelid, for instance, moves over the eye while exerting upon the eye surface a force which helps to clear the front of the eye of debris, insoluble mucin, and also expresses the oil secretions from the meibomian glands. The lower lid moves horizontally in the nasal direction and pushes debris toward both punctae, the openings that ultimately drain into the nasal cavities.

As the eyelids open the tear film is redistributed where the upper lid pulls the aqueous phase via capillary action and the lipid layer spreads as quickly as the eyelids move. Hence, eyelid movement is accordingly important in tear-film renewal, distribution, turnover, and drainage.

For a variety of reasons, the meibomian glands can become blocked, plugged, inflamed, or occluded resulting in meibomian gland dysfunction and dry eye disease. The obstruction that triggers the disease can occur anywhere within the meibomian gland, for instance, at the gland's surface or orifice preventing normal lipid secretions from flowing; in the main channel of the gland which may be narrowed or blocked; or in other locations deeper within the gland that lead to the main channel.

Treatments for blocked meibomian glands may include a number of conventional treatments. One course of treatment includes the application of soap and cleaning agents, eyelid scrubs, antiseptics, or antibiotics to reduce eyelid inflammation. Antibiotics such as tetracycline, doxycycline, minocycline, metronidazole, azithromycin, bacitracin, or erythromycin can be administered orally or topically to help regulate or improve meibomian gland lipid production. Inflammation on the surface of the eye may also be controlled with topical drugs such as corticosteroids or cyclosporine (RESTASIS®, Allergan, Inc., CA), or other anti-inflammatory compounds or immune-suppressants. Evidence suggests that ocular surface inflammation is not only associated with meibomian gland dysfunction but also with dry eye syndrome.

There exists a need for disease prognosis methods which are relatively simple to routinely use, which allow for the patient to continue their normal activities, are non-obtrusive and non-disruptive, and which also take advantage of the patient's natural physiological activities to track this treatment over time and to provide an accurate disease prognosis.

### SUMMARY OF THE INVENTION

Generally, a treatment system may include the lid treatment assemblies which may include a first lid treatment assembly for treating a first eye and a second lid treatment assembly for treating a second eye. One or both of the treatment assemblies may be electrically coupled to a controller or hub which may optionally incorporate an interactive display. A charging dock or nest for engaging with and charging controller or hub may also be optionally included as part of the treatment system. The charging dock or nest may not only include contacts or charging ports for charging the controller or hub, but it may also include storage space for storing the treatment assemblies and optionally an image capture device which may be configured to interact with the controller or hub, as described in further detail herein. In either case, one or more of these components may be omitted or included in any number of combinations as part of the treatment system.

The first lid treatment assembly may include an ear attachment such as an ear loop which may be secured around the ear of the patient, as an ear plug or insertion member which may be inserted partially within the ear canal, or any combination or mechanism which helps to temporarily hold the connector hub in position relative to the ear of the patient. A connector may be removably coupled to the connector hub and a cable may extend from the connector for electrical coupling to the controller or hub. The connector may engage with the connector hub through any number of mechanical couplings or through magnetic couplings, as described in further detail herein.

A control board such as a flex circuit may be housed within the connector hub for electrical engagement with the connector when coupled to one another. The use of a flex circuit provides the ability to incorporate electrical circuitry upon the flex circuit while maintaining flexibility of the circuit and supports. A first flexible support which may be formed as part of the flex circuit may extend or project at a distance from the flex circuit and terminate with a first heating strip positioned upon the end of the first flexible support for placement upon a first eyelid (e.g., upper lid) of the patient. A second flexible support may likewise be formed as part of the flex circuit and may also extend or project at a distance adjacent to the first flexible support such that a second heating strip is positioned upon the end of the second flexible support for placement upon a second eyelid (e.g., lower lid) of the patient. While a first and a second flexible support are shown for placement and treatment of a respective upper eyelid and lower eyelid, a single flexible support may be used in the event that a single lid is to be treated (e.g., either an upper lid or a lower lid).

The second lid treatment assembly may be similar to the first lid treatment assembly but configured for positioning over a second ear and configured for securement to a second set of eyelids. Hence, the second connector hub may be configured for engagement with a second connector which is also connectable via second cable to the connector hub. The second flex circuit may have flexible supports extend or project and terminate with heating strips similarly to the first lid treatment assembly.

The image capture device may be configured to interact (wired or wirelessly) with the controller or hub may enable the practitioner to capture images of the eye or eyes under treatment for diagnosis, tracking treatment progression over a period of time, or any number of other purposes. The image capture device may accordingly incorporate a screen for displaying the images captured by an imager. A control feature may be incorporated for controlling any number of features or commands and a handle may extend allowing for the practitioner to hold and manipulate the image capture device. A rest may be provided at the end of the handle for comfortably placing the rest upon the other arm or wrist of the practitioner during use to help hold or steady the device during image capture of the patient's eye or eyes.

When laid flat, the edges of the heating strips opposite to one another may be formed with a slight curvature or with a relatively straightened edge while the opposite edges may be curved or arcuate to follow the outer edges of the meibomian glands when applied to the respective lids. However, securing the heating strips upon the upper and lower lids of the patient may be uncomfortable for the patient due to a torsional force or moment imparted upon the heating strips (and to the underlying attached lids) by the orientation of the flexible supports. Hence, in order to increase comfort by alleviating or minimizing the torsional effects of the supports, one or more twists or bends may be imparted to the flexible supports to re-orient the heating strips by up to 90 degrees or more such that each heating strip may be re-oriented to face into apposition relative to one another. The flexible supports may be curved or bent such that the relatively straightened edges of the heating strips are now facing in the same direction towards the patient when in use and the curved or arcuate edges are now facing away from the patient. One or more additional curves or bends may be imparted directly to the heating strips as well to further conform the heating strips to the curvature of the underlying lids.

In another aspect, when diagnosing conditions such as meibomian gland dysfunction or dry eye syndrome, a first plurality of patient data may be gathered at a first time, compared to a disease data standard, and assigned a first disease severity rating. Additionally, a second plurality of patient data may be gathered at a second time, compared to the disease standard, and assigned a second disease severity rating. A first disease progression chart may include the first disease severity rating at the first time and the second disease severity rating at the second time.

The first plurality of patient data may be biometric data, physiologic data, image data, video data, survey data, and patient input data. In particular, the first plurality of patient data may be blink rate, tear meniscus height, fluorescein sodium, fluorescein staining, oscular surface disease index, tear break up time, meibography, Schirmer scores, tear volume, oscular surface staining, meibometry, rose bengal, and tear osmolarity.

Comparing the first plurality of patient data to the disease data standard may include comparing a number of dropped meibomian glands to a dropped meibomian gland average. The disease prognosis method may include gathering a third plurality of patient data at a third time, comparing the third plurality of patient data to the disease data standard, and assigning a third disease severity rating at the third time. The diagnosis method may include updating the first disease progression chart with the third disease severity rating at the third time. The disease prognosis method may include comparing the first disease progression chart to a second disease progression chart to create an abstracted disease progression chart.

The abstracted disease progression chart may be an age abstracted disease progression chart, a gender abstracted disease progression chart, a race abstracted disease progression chart, or a multifactor abstracted disease progression chart. The disease prognosis method may include compiling a plurality of disease progression charts to create a disease progression library.

A disease prognosis method may include gathering a first plurality of patient data at a first time, assigning a first data score to the first plurality of patient data, categorizing the first plurality of patient data into one of a plurality of disease states based on the first data score, and implementing a treatment plan based on the categorization.

The plurality of disease states may include a first disease state, a second disease state, a third disease state, and a fourth disease state, and each disease state may be determined at least in part by a meibomian gland area loss determination. The disease prognosis method may include selecting the first disease state when the meibomian gland area loss determination indicates no loss of meibomian glands, selecting the second disease state when the meibomian gland area loss determination indicates less than a third of the total meibomian gland area is lost, selecting the third disease state when the meibomian gland area loss determination indicates between one and two thirds of the total meibomian gland area is lost, and selecting the fourth disease state when the meibomian gland area loss determination indicates more than two thirds or more of the total meibomian gland area is lost.

The disease prognosis method may include gathering a second plurality of patient data at a second time, assigning a second data score to the second plurality of patient data, recategorizing the second plurality of patient data into one of a plurality of disease states based on the second data score, and updating the treatment plan based on the recategorization. The disease prognosis method may include adding the treatment plan and the first plurality of patient data to a disease progression library. The first plurality of patient data may include biometric data, physiologic data, image data, video data, survey data, and patient input data. In particular, the first plurality of patient data may include blink rate, tear meniscus height, fluorescein sodium, fluorescein staining, oscular surface disease index, tear break up time, meibography, Schirmer scores, tear volume, oscular surface staining, meibometry, rose bengal, and tear osmolarity.

In one aspect, a system for treating eye-related dysfunctions may generally comprise a housing defining a receiving channel, a first flexible support extending from the housing and having a first heating strip at a distal end of the first flexible support, wherein a proximal portion of the first flexible support defines a curved or bent portion which orients the first heating strip to extend in a first direction, a second flexible support extending from the housing and having a second heating strip at a distal end of the second flexible support, wherein a proximal portion of the second flexible support defines a curved or bent portion which orients the second heating strip in a second direction, wherein the first flexible support further defines a distal curved or bent portion proximal to the first heating strip and the second flexible support further defines a distal curved or bent portion proximal to the second heating strip such that the first heating strip and second heating strip are positioned to extend away from one another.

In another aspect, the system may comprise an ear attachment extending from the housing.

In another aspect, the system may comprise a flex circuit from which the first flexible support and the second flexible support each extend.

In another aspect, the first heating strip further defines a first radius of curvature.

In another aspect, the second heating strip further defines a second radius of curvature.

In another aspect, the first heating strip is rotated in a direction opposite to the second heating strip.

In another aspect, the system may comprise a connector removably attachable to the receiving channel of the housing.

In another aspect, the connector defines one or more orientation features for corresponding attachment to the receiving channel.

In another aspect, the system may comprise a magnetic feature contained within the housing for magnetic coupling to the connector.

In another aspect, the system may comprise a controller or hub in electrical communication through the housing.

In another aspect, the controller or hub is programmed to provide a treatment therapy through the first heating strip and the second heating strip.

In another aspect, the controller or hub is configured to communicate with a remote server.

In another aspect, the remote server is configured to receive treatment data from the controller or hub, analyze the received treatment data, and return a customized treatment protocol based on the analyzed treatment data.

In another aspect, the system may comprise an image capture device configured to communicate with the controller or hub.

In another aspect, the image capture device is configured to include an infrared imaging capability for capturing images under low light conditions.

In another aspect, the image capture device is configured to capture one or more images of an eye or eye-related structure.

In another aspect, the image capture device is further configured to analyze one or more captured images using machine learning algorithms to identify abnormalities in the eye.

In another aspect, the system may further comprise one or more temperature sensors located adjacent to each of the first and second heating strips, wherein the one or more temperature sensors are configured to monitor a temperature of each of the first and second heating strips and provide feedback to the controller or hub.

In another aspect, the controller or hub is programmed to adjust the temperature of each of the first and second heating strips based on feedback from the one or more temperature sensors to maintain a predetermined temperature range.

In another aspect, the system may further comprise a user interface integrated into the housing or controller, wherein the user interface is configured to allow a user to select and customize one or more treatment modes and/or durations.

In another aspect, the system may further comprise a memory within the controller or hub for storing one or more treatment histories and/or user profiles.

In another aspect, each of the first and second heating strips comprises a layer of flexible, thermally conductive material configured to conform to contours of an eye area.

In another aspect, the system may further comprise a power management system integrated within the housing, wherein the power manage system is configured to regulate power distribution to the first heating strip and the second heating strip.

In another aspect, the system may further comprise a biocompatible material coated upon the first and second heating strips.

Any of the features above may be combined in any number of combinations and such embodiments are intended to be within the scope of this description.

In another aspect, a disease prognosis method may generally comprise gathering a first plurality of patient data at a first time, comparing the first plurality of patient data to a disease data standard, assigning a first disease severity rating, gathering a second plurality of patient data at a second time, comparing the second plurality of patient data to the disease data standard, assigning a second disease severity rating, and generating a first disease progression chart including the first disease severity rating at the first time and the second disease severity rating at the second time.

In another aspect, the first plurality of patient data includes data selected from the group consisting of biometric data, physiologic data, image data, video data, survey data, and patient input data.

In another aspect, the first plurality of patient data includes gathering data selected from the group consisting of blink rate, tear meniscus height, fluorescein sodium, fluorescein staining, oscular surface disease index, tear break up time, meibography, Schirmer scores, tear volume, oscular surface staining, meibometry, rose bengal, and tear osmolarity.

In another aspect, the first plurality of patient data to the disease data standard includes comparing a number of dropped meibomian glands to a dropped meibomian gland average.

In another aspect, the method may comprise gathering a third plurality of patient data at a third time, comparing the third plurality of patient data to the disease data standard, and assigning a third disease severity rating at the third time.

In another aspect, the method may comprise updating the first disease progression chart with the third disease severity rating at the third time.

In another aspect, the method may comprise comparing the first disease progression chart to a second disease progression chart to create an abstracted disease progression chart.

In another aspect, the abstracted disease progression chart is selected from the group consisting of an age abstracted disease progression chart, a gender abstracted disease progression chart, a race abstracted disease progression chart, and a multifactor abstracted disease progression chart.

In another aspect, the method may comprise compiling a plurality of disease progression charts to create a disease progression library.

Any of the features above may be combined in any number of combinations and such embodiments are intended to be within the scope of this description.

In another aspect, a disease prognosis method may generally comprise gathering a first plurality of patient data at a first time, assigning a first data score to the first plurality of patient data, categorizing the first plurality of patient data into one of a plurality of disease states based on the first data score, and implementing a treatment plan based on the categorization.

In another aspect, the plurality of disease states includes a first disease state, a second disease state, a third disease state, and a fourth disease state, and wherein each disease state is determined at least in part by a meibomian gland area loss determination.

In another aspect, the method may comprise selecting the first disease state when the meibomian gland area loss determination indicates no loss of meibomian glands, selecting the second disease state when the meibomian gland area loss determination indicates less than a third of the total meibomian gland area is lost, selecting the third disease state when the meibomian gland area loss determination indicates between one and two thirds of the total meibomian gland area is lost, and selecting the fourth disease state when the meibomian gland area loss determination indicates more than two thirds or more of the total meibomian gland area is lost.

In another aspect, the method may comprise gathering a second plurality of patient data at a second time, assigning a second data score to the second plurality of patient data, recategorizing the second plurality of patient data into one of a plurality of disease states based on the second data score, and updating the treatment plan based on the recategorization.

In another aspect, the method may comprise adding the treatment plan and the first plurality of patient data to a disease progression library.

In another aspect, the first plurality of patient data includes data selected from the group consisting of biometric data, physiologic data, image data, video data, survey data, and patient input data.

In another aspect, gathering the first plurality of patient data includes gathering data selected from the group consisting of blink rate, tear meniscus height, fluorescein sodium, fluorescein staining, oscular surface disease index, tear break up time, meibography, Schirmer scores, tear volume, oscular surface staining, meibometry, rose bengal, and tear osmolarity.

Any of the features above may be combined in any number of combinations and such embodiments are intended to be within the scope of this description.

In another aspect, the method for disease prognosis utilizing cloud computing may comprise transmitting secure and encrypted patient-specific data from local devices directly to a cloud-based server, storing the data securely in a cloud database to ensure scalability and accessibility, processing the transmitted data using cloud-based AI algorithms to determine disease severity and progression, and updating patient records in real-time across multiple healthcare facilities to ensure uniformity in patient treatment plans.

In another aspect, the method for disease prognosis utilizing cloud computing may comprise transmitting secure and encrypted patient-specific data from local devices directly to a cloud-based server, storing the data securely in a cloud database to ensure scalability and accessibility, processing the transmitted data using cloud-based AI algorithms to determine disease severity and progression, and updating patient records in real-time across multiple healthcare facilities to ensure uniformity in patient treatment plans.

In another aspect, the method may further comprise employing machine learning models hosted on the cloud to dynamically improve disease diagnosis accuracy over time through continuous learning from new patient data.

In another aspect, the cloud-based AI algorithms may include deep learning models trained to identify patterns in complex datasets comprising various types of patient data, including imaging and biometric data, to predict disease progression stages.

In another aspect, the method may further comprise implementing a feedback loop where healthcare providers can input treatment outcomes, which are used by AI to refine and adjust predictive models for better accuracy.

In another aspect, the method for generating personalized treatment plans using AI may comprise analyzing patient data using AI algorithms to categorize disease severity, generating a treatment plan tailored to the individual patient's disease state and predicted progression, and continuously updating the treatment plan based on real-time data analysis and machine learning insights to adapt to changes in the patient's condition.

In another aspect, the method may further comprise utilizing natural language processing (NLP) algorithms to interpret and integrate clinical notes and patient-reported outcomes into the treatment planning process.

In another aspect, the AI-driven analysis includes using predictive analytics to forecast disease progression based on historical data and similar patient profiles, and enhancing the decision-making process for early interventions.

In another aspect, the method for collaborative disease management utilizing cloud and AI technologies may comprise sharing access to a cloud-based platform among different healthcare providers to facilitate a coordinated treatment approach and using AI to recommend adjustments to treatment plans based on data aggregated from multiple providers and patients, promoting an integrated care model.

In another aspect, the method may further comprise enabling real-time consultation and updates to treatment plans through a cloud interface, and allowing for immediate expert feedback and adjustments based on AI recommendations.

In another aspect, the method for a cloud-based disease prognosis platform may comprise a data collection module for acquiring patient-specific data, a cloud infrastructure for data storage and processing, an AI module implemented on the cloud for analyzing the data and determining disease severity, and a user interface module that allows healthcare providers to access and interact with the AI-generated insights and treatment recommendations.

### BRIEF DESCRIPTION OF THE DRAWINGS

FIG. 1A shows a cross-sectional side view of an upper eyelid and an example of the location of a meibomian gland.

FIG. 1B shows a front view diagram of meibomian gland distribution in human eyelids having the upper eyelid and lower eyelid in a closed position, such as when the patient blinks, and the alignment of the meibomian glands over both the upper and lower eyelids.

FIG. 1C shows a perspective view of a patient's eye in the open position to illustrate how the meibomian glands are typically aligned relative to one another when the patient's eye is opened.

FIG. 2A shows a front view of a patient's eye in a closed position with an example of treatment strips which adhere onto the upper or lower eyelids (or both) and where the strips are sized or contoured for placement directly over the meibomian glands located in the underlying eyelids.

FIG. 2B shows the treatment strips of FIG. 2A illustrating how the strips may remain adhered to the patient skin while allowing for the eyelids to retract and allow for the patient to continue blinking while viewing normally out of the eye. While the strips may be applied from eyelid margin to eyelid crease, they may alternatively flex or accordion and/or compress during blinks to prevent impairment of normal blinking and maximize comfort.

FIG. 3 shows one variation of a treatment system which may be used to treat a number of eye-related conditions.

FIGS. 4A and 4B show perspective views of the first lid treatment assembly.

FIGS. 5A and 5B show side and front views of the connector coupled to the cable via a connector interface which may extend for connection to a yoke.

FIGS. 6A and 6B show respective front and perspective views of the hub connector.

FIG. 7 shows an exploded perspective assembly view of lid treatment assembly.

FIGS. 8A and 8B show detail views of the receiving channel and examples of the orientation features.

FIG. 9A shows a side view of the flex circuit removed from the hub for clarity.

FIG. 9B shows a side view the lid treatment assembly having one or more curves or bends imparted to the flexible connectors and heating strips.

FIGS. 10A and 10B show perspective views of lid treatment assembly with and without the housing of connector hub to illustrate the positioning and plane of the flex circuit relative to the plane defined by the flexible supports and heating strips.

FIGS. 11A to 11C show respective side, top, and bottom views of lid treatment assembly.

FIG. 11D shows an end view of the heating strips.

FIGS. 12A and 12B illustrate top and detailed top views of a lid treatment assembly to illustrate one variation for how the heating strip may be configured.

FIGS. 13A and 13B illustrate schematic top views of how the imparted curves and twists along the flexible supports and heating strips may help to facilitate patient treatment.

FIG. 14 shows an example of the lid treatment assemblies secured to each ear of the patient.

FIGS. 15A and 15B illustrate perspective front and back views of one variation of the controller or hub.

FIG. 15C shows a front perspective view of the charging dock or nest.

FIG. 16 shows an exploded perspective assembly view of one variation of the controller or hub.

FIGS. 17A and 17B show perspective rear and front views of one variation of the image capture device.

FIG. 17C shows a variation where the image capture device may incorporate a stand.

FIG. 18A shows an image capture device having a captured image.

FIG. 18B shows an example of a captured image.

FIGS. 19A to 19C illustrate images of various conditions of the patient's eye aside from the meibomian glands.

FIG. 20A illustrates one example for how a meibography system may be implemented using the controller or hub in combination with the image capture device.

FIG. 20B illustrates another example functionally of how meibography may be implemented.

FIG. 21 illustrates a flow diagram of how the grading system may be implemented in one example.

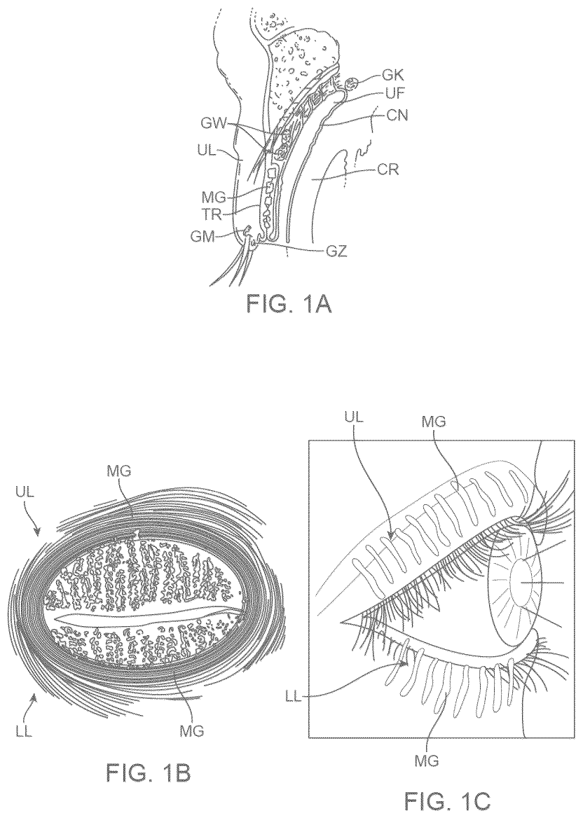

### DETAILED DESCRIPTION OF THE INVENTION

In treating various eye-related conditions such as meibomian gland dysfunction (MGD), which is commonly associated with the evaporative form of dry eye syndrome (DES), a patch, strip or thin adhesive device can be affixed to the skin of the upper and/or lower eyelids to deliver or absorb heat or other forms of energy, pressure, drugs, moisture, etc. (alone or in combination) to the one or more meibomian glands contained within the underlying skin as well as the muscular structures surrounding the eyes. In particular, the treatment strip or strips may be configured and sized specifically for placement over one or more targeted meibomian glands contained within the skin of the upper and/or lower eyelids. The application of thermal therapy, e.g., heating or cooling, can cross the eyelids quite easily as the eyelids are generally the thinnest skin found on the human body and the tissue is highly vascularized. With the root of the eyelid located proximally and the eyelid margin located distally, the net arterial flow of blood flows from proximal to distal. So wherever these treatment strips are placed, the heating or cooling therapy may easily be carried throughout the eyelid and any structures contained therein, e.g., meibomian glands MG, lacrimal glands LG, gland of Zeis GZ, gland of Moll GM, gland of Wolfring GW, gland of Kraus GK, etc.

Moreover, because the eyelid is so thin, the heating or cooling therapy can be transmitted to the ocular surface and the eye itself (described in further detail below). Thus, the therapy can impart energy to the conjunctiva, goblet cells, episcleral vasculature, cornea, aqueous humor, iris, ciliary body, and possibly the retina, choroid, optic nerve, anterior vitreous, and lens. Thus, any thermal therapy by the treatment strips may also impact and be used to treat ocular surface disorders and anterior segment diseases, e.g., conjunctivitis, keratitis, keratopathy, iritis, cyclitis, glaucoma, cataract, etc. Also, there may be use in the postoperative state-like after LASIK, PRK, or cataract or corneal surgery or other ocular, peri-ocular, intraocular, or eyelid surgery, as described in further detail below.

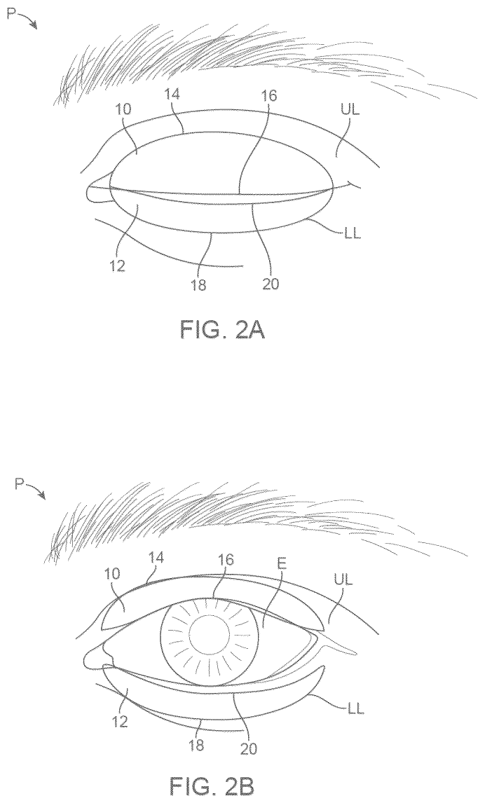

As shown in the front view of FIG. 2A and FIG. 2B, one variation of such treatment strips may be seen as being adhered temporarily upon the upper eyelid UL and lower eyelid LL over an eye of a patient P when closed for illustrative purposes. The contoured upper strip 10 may be sized for adherence directly upon the skin of the upper eyelid UL such that the strip 10 has a configuration and shape which follows the location of the one or more meibomian glands contained within the underlying skin of the upper eyelid UL. Likewise, the contoured lower strip 12 may also have a configuration and shape which follows the location of the one or more meibomian glands contained within the underlying skin of the lower eyelid LL. In other variations, the contoured strip may stop at the eyelid crease or cross over it as described in other variations below.

The upper strip 10 may thus have an upper curved or arcuate periphery 14 which is shaped to extend and follow the upper (or superior) border of the meibomian glands (such as along or up to the upper eyelid crease) while the straightened periphery 16 of the lower edge may be shaped to extend and follow the lower (or inferior) border of the meibomian glands such as along the free margin of the upper eyelid UL. The lower strip 12 may similarly have an upper straightened periphery 20 to extend and follow the upper (or superior) border of the meibomian glands along the free margin of the lower eyelid LL and a lower curved or arcuate periphery 18 to extend and follow the lower (or inferior) border of the meibomian glands along the lower eyelid LL (such as along or up to the lower eyelid crease). The use of the terms lower and upper herein refer to the periphery of the treatment strips when placed upon the patient P (human or animal) and are used herein for descriptive purposes.

While the treatment strips 10, 12 are both shown adhered upon the respective upper eyelid UL and lower eyelid LL, the strips 10, 12 may be used individually for placement upon only the upper eyelid UL or only the lower eyelid LL depending upon the desired treatment. Moreover, the lengths of the treatment strips 10, 12 may also be varied to target individual meibomian glands for providing a targeted treatment, if desired, and as described in further detail herein.

While the treatment strips 10, 12 are shown placed upon the closed eyelids of the patient P, the strips 10, 12 are arc-shaped or flexible enough to assume the curvature of the patient's eyelid margin and may be long enough to cover some or all of the underlying meibomian glands in the tarsal plate. While the treatment strips 10, 12 may be sized generally, they may also be custom made or sized for a specific individual's eyelid dimensions or shaped to optimize adhesion and/or comfort and/or stability. Generally, the treatment strips 10, 12 may have a length anywhere from about 1 mm to 50 mm depending upon the desired treatment length as well as the anatomical considerations of the patient since the typical palpebral fissure length in an adult is about 27 mm to 30 mm. Thus, to cover as many as all of the meibomian glands, the treatment strips 10, 12 may be sized to have length of, e.g., 25 mm to 30 mm, or if sized to cover just beyond all the meibomian glands, a length of, e.g., 30 mm to 50 mm (or more if needed to optimize coverage/adhesion/comfort/stability). Moreover, one or both treatment strips 10, 12 can have a width ranging anywhere from about 1 mm to 25 mm since the typical eyelid crease in a Caucasian male is about 8 mm to 9 mm above the eyelid margin while in Caucasian females it is about 9 mm to 11 mm above the eyelid margin (or more if needed for adhesion/comfort and potentially increased efficacy from heating or cooling the inbound blood flow). Customization enables it to fit any particular anatomy, race, ethnicity, etc. Moreover, the treatment strips may be manufactured with varying levels of flexibility to accommodate the ergonomics of the eyelid and eyelid blink for optimal comfort and minimal obtrusiveness or movement.

Because of the specific contoured sizes and flexibility of the treatment strips 10, 12, the treatment strips may be placed upon the patient P by the patient himself/herself for consumer use or by a healthcare provider to apply therapy to the underlying meibomian glands allowing the patient's eyes to be opened and closed normally, as shown in FIG. 2B, without interference from one or both treatment strips. While the strips may be applied from eyelid margin to eyelid crease, they may alternatively flex or accordion and/or compress during blinks to prevent impairment of normal blinking and maximize comfort.

Typical treatment patches, such as for application of a warm compress, are generally sized for placement over the entire eye or eyes such that the patient is unable to open their eyes or blink during a treatment session. Yet, because of the strong association between DES and MGD (for instance, MGD includes the spectrum of MGD, meibomitis, blepharitis, and ocular rosacea), natural blinking by an individual is the mechanism by which meibomian gland secretions are normally released onto the eyelid margin and over the tear. In the absence of blinking, the oil contained within the meibomian glands remain unexpressed within the glands' terminal ducts and fail to contribute to distribution of the oily layer upon the tears.

Accordingly, the treatment strips 10, 12 contoured size, shape, and flexibility allow for treatment to occur while also allowing for the patient to have one or both eyes remain opened such that normal, physiologic blinking can proceed during the course of treatment. Rather than relying on an application of any type of external force to express the oil or obstruction from the glands, the treatment strips 10, 12 take advantage of the eye's natural mechanism for clearing oil from the meibomian glands via blinking. Hence, the treatment strips 10, 12 may be adhered in place for treatment without any further intervention by the patient or healthcare provider such that the treatment strips 10, 12 may apply, e.g., heat energy, to melt or liquefy any waxy or solid meibomian gland obstructions while the eyes remain unobstructed and are allowed to blink naturally. The treatment strips 10, 12 thus allow for the natural blinking to help clear the glands of the heat-treated softened obstructions before they have re-solidified unlike other treatments which require that the patient keep their eyes closed or obstructed during the course of a treatment and prevent or inhibit the patient from blinking. Delivery of heat may also increase blood flow by promoting vasodilation as increased delivery of blood can affect metabolism, temperature of other tissues, may have effects on inflammation, and can thereby improve tissue function or recovery.

Because some patients have obstructions or occlusions in their meibomian glands that may not sufficiently melt, loosen, or soften without attaining heightened temperatures at the meibomian glands, the treatment strips 10, 12 may apply heat or other treatments to the surface of the eyelids for a significant period of time for relatively longer treatment times and at higher treatment temperatures because of the ability of the treatment strips 10, 12 to remain attached to the patient during any given period throughout the day. Treatment strips may be relatively transparent or skin toned, and thereby inconspicuous, to allow for normal user/activity function throughout the treatment. Patients can assume their daily activities with their eyes open and eyes blinking and with the comfort of a strip-based treatment. Moreover, patients can affix the treatment strips as many times as needed throughout the day, week, month, etc. until dry eye symptoms subside. This increases the frequency of treatment, convenience of treatment, and thus efficacy of treatment.

Because of the prolonged treatment times, the application of a separate force beyond the application of the strips may not be needed so long as the patient is able to continue blinking during the course of treatment. Moreover, the treatment frequency may be adjusted or varied depending upon the severity of the condition to be treated. One example for potential treatment frequency may include application of one or both strips, e.g., up to six times per day for ten minutes or up to an hour or more for each treatment. Moreover, because the treatment strips are positioned over the meibomian glands which overlie the ocular surfaces, the application of the heating therapy may also indirectly heat the ocular surface as well and may further reduce any chronic ocular surface inflammation, chronic conjunctival inflammation, or corneal neovascularization.

Aside from heating of the ocular surface, heat therapy may also optionally be used to potentially provide for indirect heating through the ocular surface as well for heating of the retina to provide a thermal therapy to limit inflammation and neovascularization which are underlying conditions for diseases such as age-related macular degeneration (AMD), retinal vascular occlusions, retinal neovascularization, glaucoma, retinal degenerations and dystrophies, and Diabetic Retinopathy.

While the treatment strips 10, 12 may be used throughout the day to take advantage of the patient's physiologic blinking, the treatment strips 10, 12 may also be used while the patient is resting or sleeping or while the patient simply maintains their eyes closed. The treatment strips 10, 12 may applied as a single-use treatment or they may be configured to be robust enough as a re-usable device.

Additional details may be found in the following U.S. patents each of which is incorporated herein by reference in its entirety and for any purpose herein: U.S. Pat. Nos. 9,510,972; 9,642,743; 9,724,230; 9,844,459; 10,052,226; 10,772,758; 10,925,765; 10,973,680; 11,285,040. Further details may also be found in the following U.S. patent publications each of which is incorporated herein by reference in its entirety and for any purpose herein: 2020/0078211; 2020/0405534; 2021/0022914; 2021/0052216; 2021/0177647; 2021/0177648; 2022/0168136.

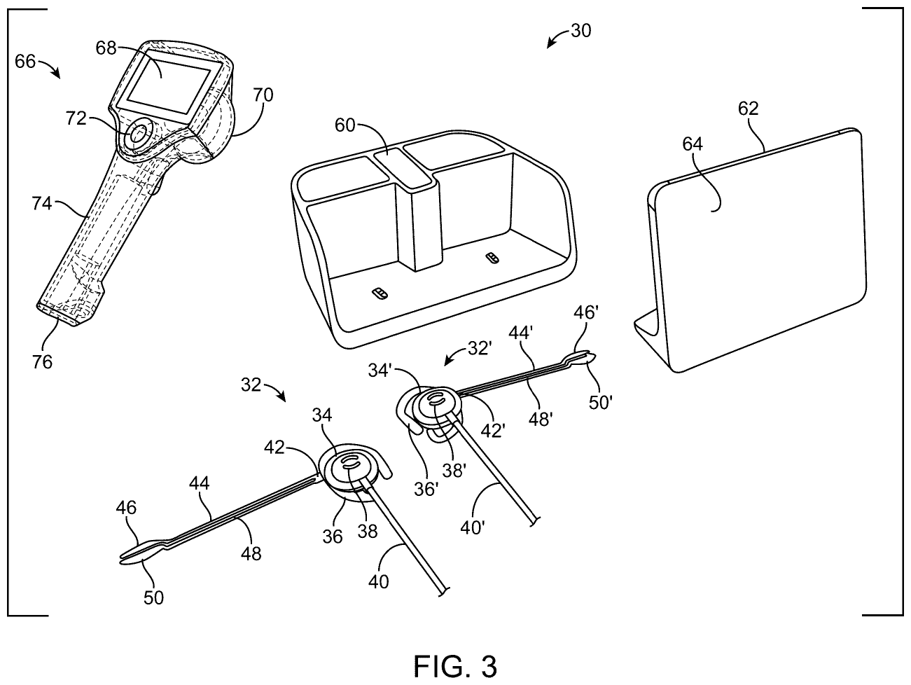

One variation of a treatment system 30 which may be used to treat a number of eye-related conditions such as dry eye syndrome is shown in the assembly view of FIG. 3. Generally, the treatment system 30 may include the lid treatment assemblies which may include a first lid treatment assembly 32 for treating a first eye and a second lid treatment assembly 32′ for treating a second eye. One or both of the treatment assemblies 32, 32′ may be electrically coupled to a controller or hub 62 which may optionally incorporate an interactive display 64. A charging dock or nest 60 for engaging with and charging controller or hub 62 may also be optionally included as part of the treatment system 30. The charging dock or nest 60 may not only include contacts or charging ports for charging the controller or hub 62, but it may also include storage space for storing the treatment assemblies 32, 32′ and optionally an image capture device 66 which may be configured to interact with the controller or hub 62, as described in further detail herein. Alternatively, the dock or nest 60 may be configured as a docking station which is portable and battery powered for docking or storing any of the various components of the treatment system 30. In either case, one or more of these components may be omitted or included in any number of combinations as part of the treatment system 30.

Turning now to the lid treatment assemblies 32, 32′, one or both assemblies 32, 32′ may be included but in either case, the assemblies 32, 32′ may resemble one another and function similarly to one another while each being configured for treating either a left eye or a right eye. Further details of the functionality of the lid treatment assemblies may also be seen in the patents and patent publications incorporated hereinabove.

The first lid treatment assembly 32 is shown as having a connector hub 34 and an ear attachment 36 such as an ear loop which may be secured around the ear of the patient, as an ear plug or insertion member which may be inserted partially within the ear canal, or any combination or mechanism which helps to temporarily hold the connector hub 34 in position relative to the ear of the patient. A connector 38 may be removably coupled to the connector hub 34 and a cable 40 may extend from the connector 38 for electrical coupling to the controller or hub 62. The connector 38 may engage with the connector hub 34 through any number of mechanical couplings or through magnetic couplings, as described in further detail herein.

A control board such as a flex circuit 42 may be housed within the connector hub 34 for electrical engagement with the connector 38 when coupled to one another. The use of a flex circuit 42 provides the ability to incorporate electrical circuitry upon the flex circuit 42 while maintaining flexibility of the circuit 42 and supports. A first flexible support 44 which may be formed as part of the flex circuit 42 may extend or project at a distance from the flex circuit and terminate with a first heating strip 46 positioned upon the end of the first flexible support 44 for placement upon a first eyelid (e.g., upper lid) of the patient. A second flexible support 48 may likewise be formed as part of the flex circuit 42 and may also extend or project at a distance adjacent to the first flexible support 44 such that a second heating strip 50 is positioned upon the end of the second flexible support 48 for placement upon a second eyelid (e.g., lower lid) of the patient. While a first and a second flexible support 44, 48 are shown for placement and treatment of a respective upper eyelid and lower eyelid, a single flexible support may be used in the event that a single lid is to be treated (e.g., either an upper lid or a lower lid).

The second lid treatment assembly 32′ is shown and may be similar to the first lid treatment assembly 32 but configured for positioning over a second ear and configured for securement to a second set of eyelids. Hence, the second connector hub 34′ may be configured for engagement with a second connector 38′ which is also connectable via second cable 40′ to the connector hub 34. The second flex circuit 42′ may have flexible supports 44′, 48′ extend or project and terminate with heating strips 46′, 50′ similarly to the first lid treatment assembly 32. Further details with respect to the lid treatment assemblies are provided hereinbelow.

The image capture device 66 may be configured to interact (wired or wirelessly) with the controller or hub 62 may enable the practitioner to capture images of the eye or eyes under treatment for diagnosis, tracking treatment progression over a period of time, or any number of other purposes. The image capture device 66 may accordingly incorporate a screen 68 for displaying the images captured by an imager 70. A control feature 72 may be incorporated for controlling any number of features or commands and a handle 74 may extend allowing for the practitioner to hold and manipulate the image capture device 66. A rest 76 may be provided at the end of the handle 74 for comfortably placing the rest 76 upon the other arm or wrist of the practitioner during use to help hold or steady the device 66 during image capture of the patient's eye or eyes. Further details of the image capture device 66 are provided hereinbelow.

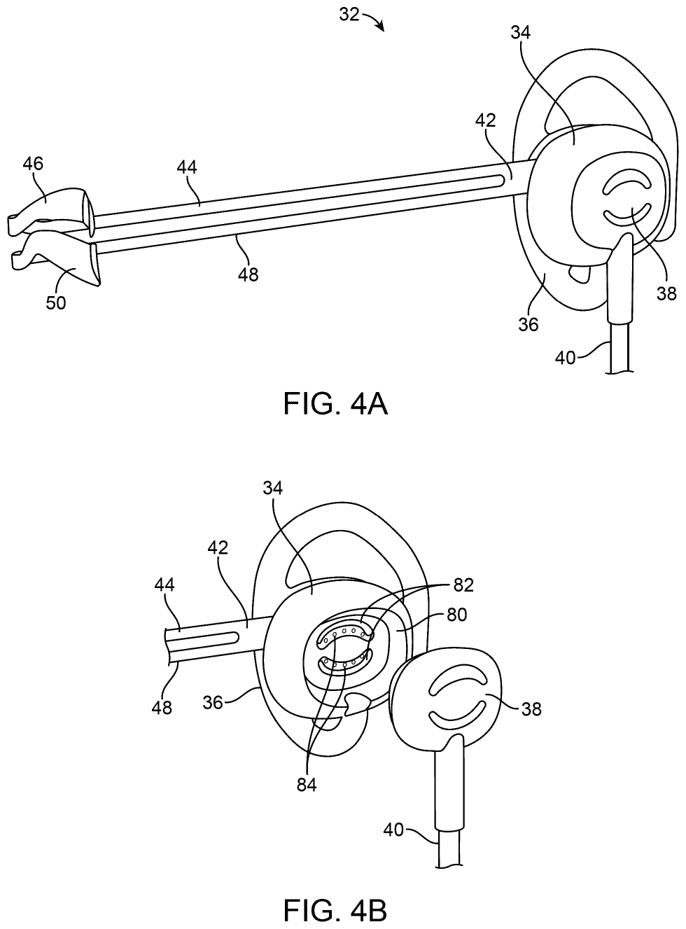

Turning back to the lid treatment assemblies, FIGS. 4A and 4B show perspective views of the first lid treatment assembly 32 for illustrative purposes. While the second lid treatment assembly 32′ is not shown, the same principles apply similarly. The connector hub 34 may be removably coupled to connector 38 using any number of mechanical engagements or magnetic engagements. To ensure that connector 38 may be attached quickly while also ensuring electrical connection between the two, one or more orientation features 82 may be incorporated within the connector receiving channel 80 so that corresponding orientation projections on the connector 38 may engage within the orientation features 82 in a keyed manner so that the connector 38 mates within the receiving channel 80 in a consistent and secure manner. The keyed engagement also helps to ensure that the power/data connectors 84 within the receiving channel 80 are mated in a corresponding manner with the connectors within the connector 38.

Furthermore, the ability to remove the connector 38 from the lid treatment assembly 32 allows for the connector 38 and cables to be reused while the lid treatment assemblies may be disposed. Additionally, the ear attachment 36 feature also allows for the connector hub 34 to be positioned relative to the corresponding eyelids in a consistent manner without having to adhere the connector hub 34 to the skin of the patient which may introduce variability in the connector hub positioning relative to the eyelids. This variability may further introduce differences in angling and/or length of the heating strip support members which extend from the connector hub 34. Hence, maintaining a consistent distance, positioning, and orientation between the connector hub 34 and eyelid positioning may allow for a consistent treatment and comfort.

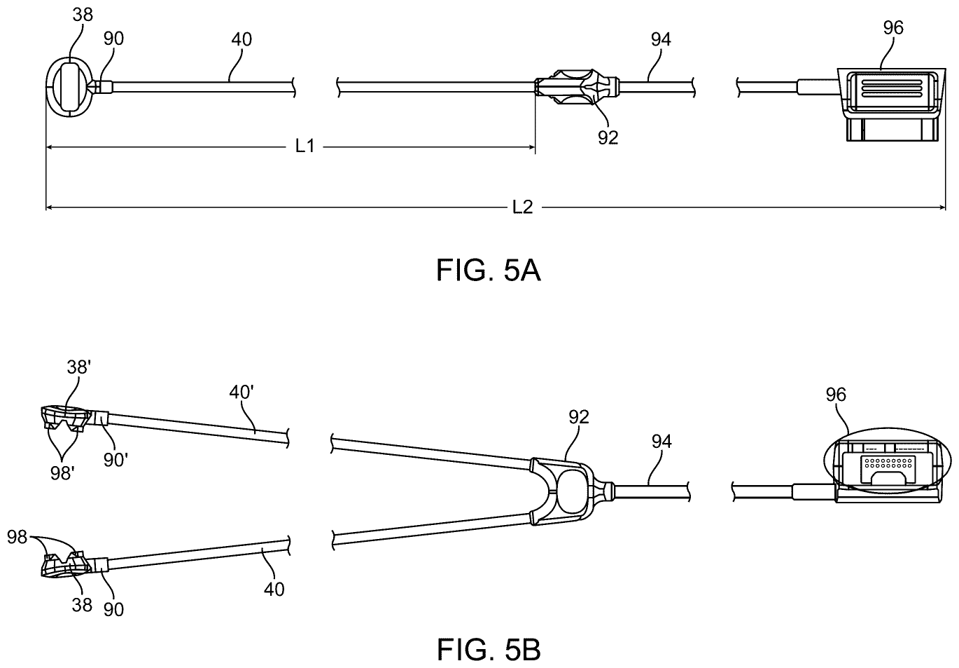

The connector 38 may be coupled to the cable 40 via a connector interface 90 which may extend for connection to a yoke 92 which may receive a second cable 40′ from the second connector 38′ which may be coupled to the second cable 40′ via a second connector interface 90′, as shown in the side and front views of FIGS. 5A and 5B. The two individual cables 40, 40′ from the respective connectors 38, 38′ may be coupled to the yoke 92 such that a single cable 94 may extend from the yoke 92 for connection to a single hub connector 96 which may be removably coupled to the controller or hub 62.

To accommodate movement by the patient when the connectors 38, 38′ are coupled to the respective connector hubs 34, 34′ during treatment, the first and second cables 40, 40′ may extend at a length L1 (e.g., 23 cm+/−10 cm) from the connector end to the yoke 92. The overall length of the cable from the connector end to the hub connector 96 may extend at a length L2 (e.g., 180 cm+/−50 cm) although the lengths L1, L2 may be varied to any number of distances depending upon the desired length.

Additionally, the orientation projections 98, 98′ which correspond to orientation features 82, 82′ within the connector receiving channels 80, 80′, as described above, may also be seen in the front view of FIG. 5B.

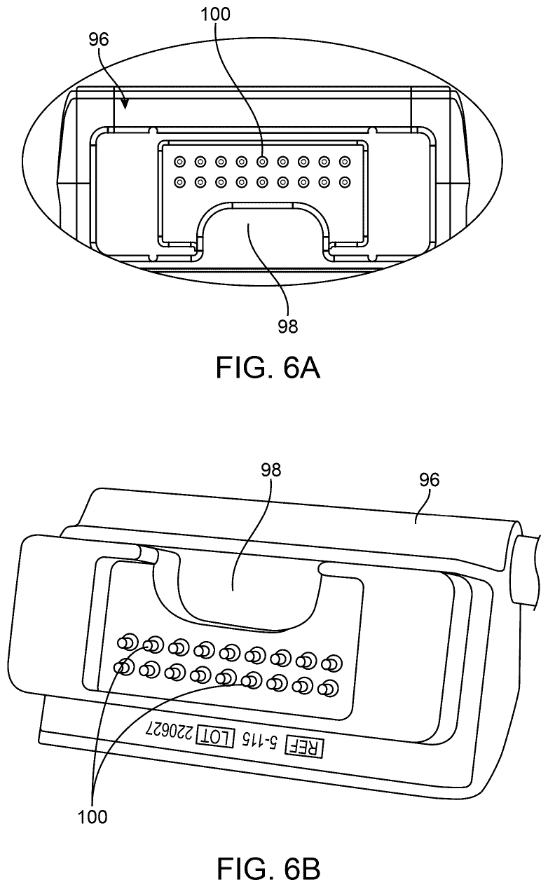

FIGS. 6A and 6B show respective front and perspective views of the hub connector 96 to illustrate how the connector 96 may incorporate the power/data connectors 100 for transferring data and power between the lid treatment assemblies 32, 32′, through the cables 40, 40′, and to the controller or hub 62. The connector 96 may also incorporate one more orientation feature 98 which enables the consistent orientation when coupling the connector 96 to the controller or hub 62.

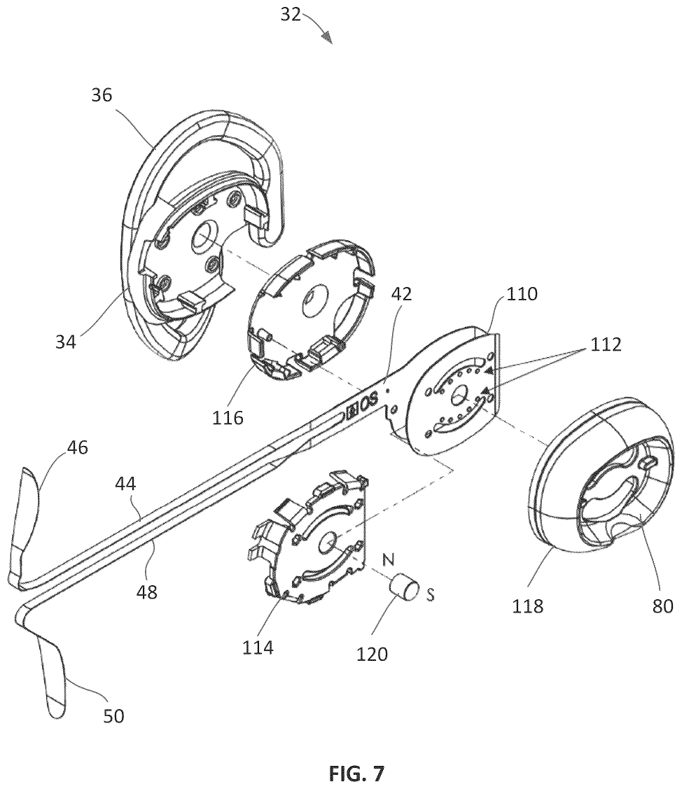

To illustrate how the lid treatment assemblies may incorporate the flex circuit within, FIG. 7 shows an exploded perspective assembly view of lid treatment assembly 32. The connector hub 34 is shown with ear attachment 36 extending from the hub 34. An outer housing 118 which receives the connector 38 may contain an inner housing 116 which contains the flex circuit 42 which sandwiches the flex circuit 42 between the outer housing 118. The connector hub 34 may also contain a mid-layer housing 114 which retains a magnetic feature 120 for coupling to the connector 38. The flex circuit 42 may extend within the housing and form a bend or curve 110 to expose and align a number of pin connector targets 112 on the flex circuit 42 which are engaged into electrical communication with the power/data connectors 84.

The magnetic feature 120 may include a permanent magnet or any other type of magnet which allows for the magnetic engagement with a metallic portion or a corresponding magnet in the connector 38. The use of such a magnet may allow for the quick connect and disconnect of the connector 38 from the receiving channel 80. Furthermore, the magnetic feature 120 may also allow for the ease of coupling as the magnetic attraction may still allow for the coupling of the connector 38 even if the connector 38 is off orientation by, e.g., 30 to 40 degrees. The attraction of the magnetic feature 120 may enable the self-alignment and connection due also to the orientation features described herein.

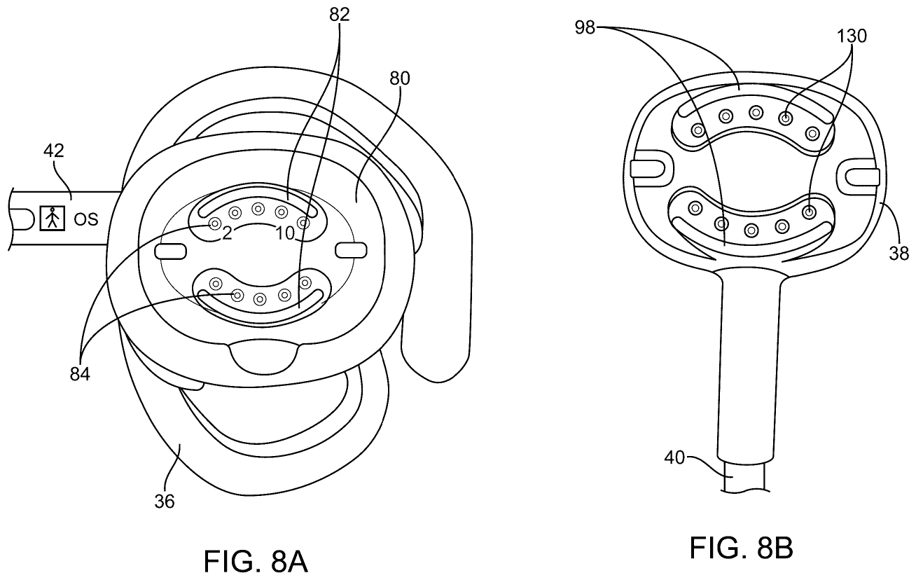

FIGS. 8A and 8B show detail views of the receiving channel 80 and examples of the orientation features 82 which in this variation are shown as curved receiving channels which follow the positioning of the power/data connectors 84. FIG. 8B shows a detail view of the connector 38 illustrating how the one more orientation feature 98 may be configured as curved projections which correspond to the orientation features 82 within the receiving channel 80. The connector 38 also shows the power/data connectors 130 which are also aligned in a curved orientation such that the magnetic feature 120 contained within the connector hub 34 may draw the connector 38 into engagement and the insertion of the orientation features 98 may force the alignment of the connector 38 relative to the receiving channel 80 and the subsequent electrical engagement between the power/data connectors 130 and the power/data connectors 84.

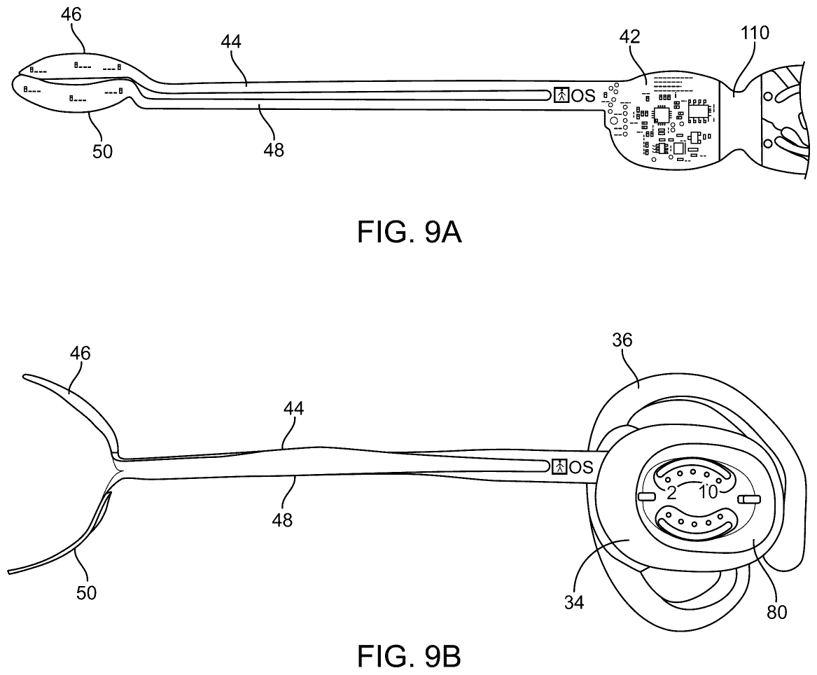

With the first and second heating strips 46, 50 and first and second flexible supports 44, 48 extending from the flex circuit 42, the heating strips 46, 50 may be seen to lay flat or within the same plane defined by the flex circuit 42, as shown in the side view of FIG. 9A of the flex circuit 42 removed from the hub for clarity. When laid flat, the edges of the heating strips 46,50 opposite to one another may be formed with a slight curvature or with a relatively straightened edge while the opposite edges may be curved or arcuate to follow the outer edges of the meibomian glands when applied to the respective lids. However, securing the heating strips 46, 50 upon the upper and lower lids of the patient may be uncomfortable for the patient due to a torsional force or moment imparted upon the heating strips 46, 50 (and to the underlying attached lids) by the orientation of the flexible supports 44, 48. Hence, in order to increase comfort by alleviating or minimizing the torsional effects of the supports 44, 48, one or more twists or bends may be imparted to the flexible supports 44, 48 to re-orient the heating strips 46, 50 by 90 degrees or more such that each heating strip 46, 50 may be re-oriented to face into apposition relative to one another. The flexible supports 44, 48 may be curved or bent such that the relatively straightened edges of the heating strips 46, 50 are now facing in the same direction towards the patient when in use and the curved or arcuate edges are now facing away from the patient. One or more additional curves or bends may be imparted directly to the heating strips 46, 50 as well to further conform the heating strips to the curvature of the underlying lids, as shown in FIG. 9B.

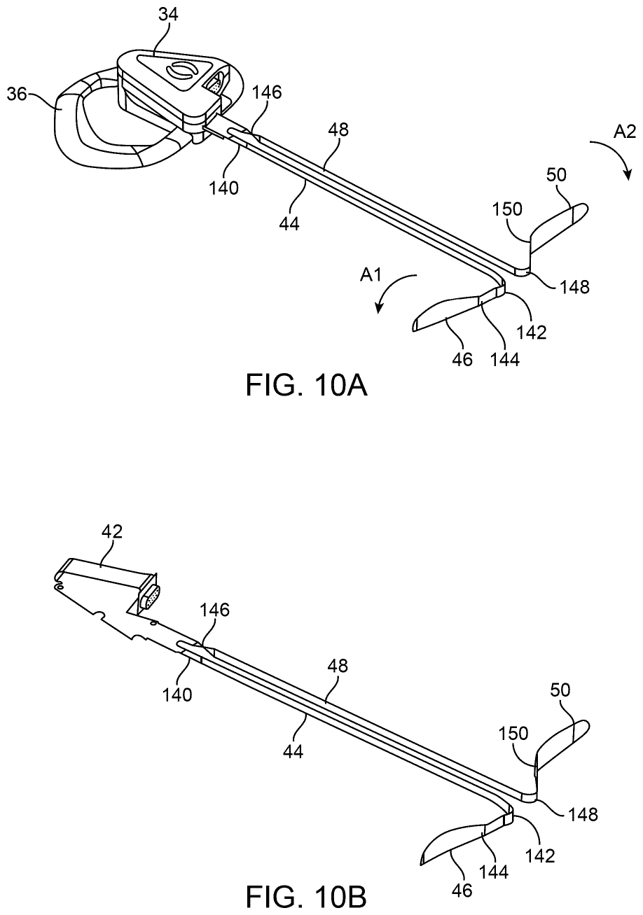

FIGS. 10A and 10B show perspective views of lid treatment assembly 32 with and without the housing of connector hub 34 to illustrate the positioning and plane of the flex circuit 42 relative to the plane defined by the flexible supports 44, 48 and heating strips 46, 50. Generally, the flexible supports 44, 48 may have a proximal curve or bend 140, 146 which reorients the direction of the heating strips 46, 50. The proximal curve or bend 140, 146 may be formed as close proximally to the static portion of connector hub 34. The inclusion of a variable area moment (e.g., twist) may allow for anatomical conformance and may also accommodate dynamic movement (e.g., blinking by the patient) during a treatment procedure. For instance, first flexible support 44 may be curved or bent at proximal curve or bend 140 such that the heating strip 46 is oriented at 0 to 90 degrees or more in the direction A1 and second flexible support 48 may be curved or bent at proximal curve or bend 146 such that the heating strip 50 is oriented at 0 to 90 degrees or more in the direction A2 opposite to the direction A1, as illustrated, such that the outer curved portions of each heating strip 46, 50 face away from the patient. From the perspective of looking down from the connector hub 34 towards the heating strips 46, 50, the first flexible support 44 may be curved or twisted in a clockwise direction A1 while the second flexible support 48 may be curved or twisted in an opposite counter-clockwise direction A2.

The portion of the first flexible support 44 may be further curved or bent along the portion 142 just proximal to the heating strip 144 in a first direction facing away from heating strip 50. Similarly, second flexible support 50 may be curved or bent along the portion 148 just proximal to the heating strip 150 in a second direction opposite to the first direction and that of portion 142 such that the distal tips of each heating strip 46, 50 face away from one another. The heating strips 46, 50 may each be further curved or bent 144, 150 such that the radius of curvature faces away from the flexible supports 44, 48. The curvature or bend 144, 150 may allow for the heating strips 46, 50 to better conform to the underlying lid tissue surface to provide improved comfort. Furthermore, while a single curve or bend is shown along the curved or bent portions 144, 150, multiple twists or bends may be utilized is desired.

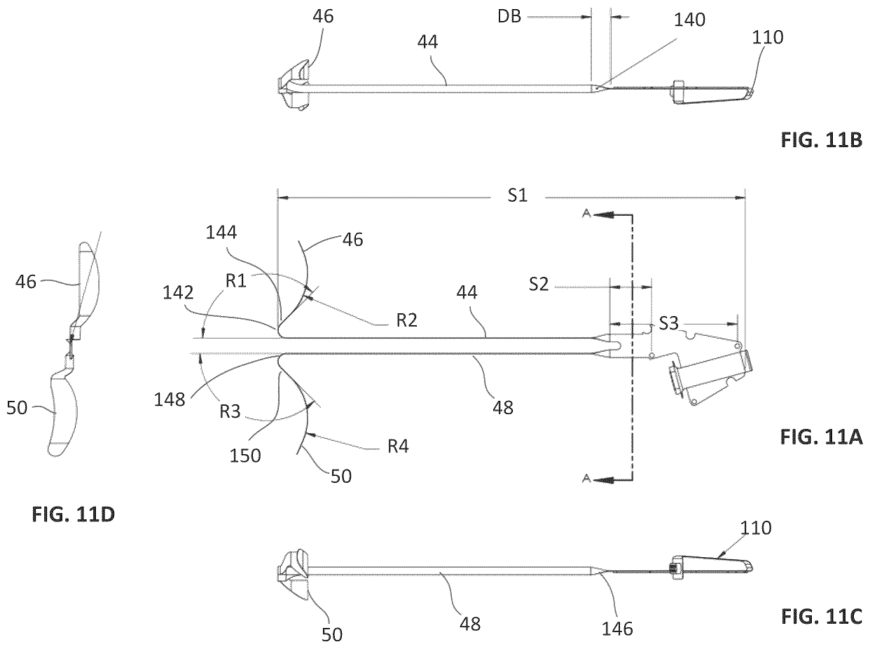

FIGS. 11A to 11C show respective side, top, and bottom views of lid treatment assembly 32 and FIG. 11D shows an end view of the heating strips 46, 50 to further illustrate the various curves and bends imparted. In the variation shown, the proximal curve or bend 140 may curve the flexible support 44 over a distance DB, e.g., 6.3 to 6.4 mm, as shown in FIGS. 11B and 11C. The overall length S1 of the heating strip assembly from the end of the flexible support 44 to the proximal end of the flex circuit 42 may be varied to provide for enough length to accommodate the ear to eyelid distance of a range of patients anatomies. The overall length S1 may be sufficient long, e.g., 155 mm, to ensure that the lid treatment assembly may be comfortably applied to the patient. The length S2 along the flex connector extending from the hub housing may be, e.g., 13-14 mm, while the length S3 along the flex connector within the hub housing may be, e.g., 42-43 mm.

While the proximal curve or bend 140, 146 may orient the flexible support 44, 48 to be orthogonal or transverse relative to the flex circuit, the curved or bent portion 142, 148 may impart a curvature having a first radius R1, e.g., 135 degrees, of heating strip 46 and second radius R2, e.g., 135 degrees, of heating strip 46 such that the heating strips 46, 50 are curved back away from one another. FIG. 11A illustrates a side view and FIG. 11D illustrates an end view of the heating strips 46, 50 extending away from one another. Each of the heating strips 46, 50 may also each have a curvature imparted, as shown in FIG. 11A, whereby the radius of curvature faces away from the flexible supports 44, 48. The first radius of curvature R3, e.g., 19-20 mm, of heating strip 50 is shown to mirror the second radius of curvature R3, e.g., 19-20 mm, of heating strip 50 and may help to reduce adhesive shear against the skin surface of the eyelid when the heating strips are secured to the skin surface for treatment.

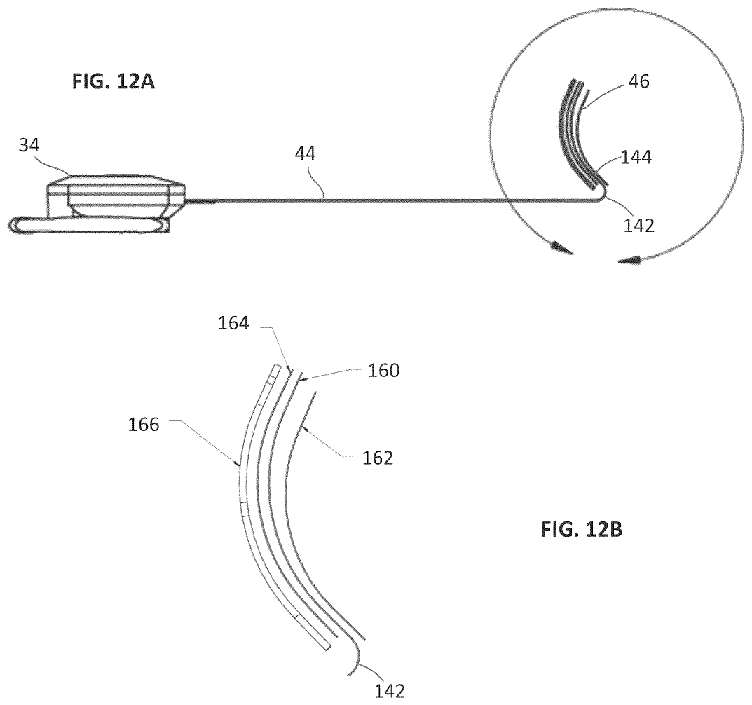

FIGS. 12A and 12B illustrate a top view of a lid treatment assembly 32 and a detailed top view in FIG. 12B to illustrate one variation for how the heating strip may be configured. As shown, the flex circuit 160 may extend and form a platform for the heating strip 46. An adhesive layer 162 such as a pressure sensitive adhesive may be applied along the concave surface of the heating strip for adhering the strip to the underlying lid surface. A release liner may be applied over the adhesive layer 162 for storage purposes and the release liner may be removed prior to application of the adhesive layer 162 to the patient's lid surface. A copper backing layer 164 having a thickness, e.g., 0.05-0.20 mm, may be applied to the surface of the flex circuit 160 opposite to the adhesive layer 162 in order to provide an even thermal distribution over the area of the heating layer. A backing layer 166 such as a foam backing may be provided upon the copper backing layer 164 to prevent exposure of the heated layer to the patient or practitioner.

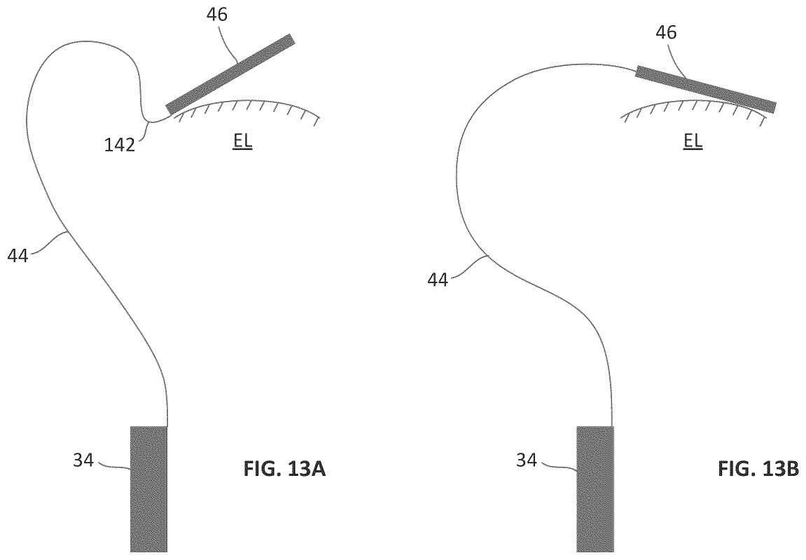

FIGS. 13A and 13B illustrate schematic top views of how the imparted curves and twists along the flexible supports and heating strips may help to facilitate patient treatment. FIG. 13A shows an example of how the twisted flexible support 44 and curved heating strip 46 help to conform the heating strip 46 against the skin surface of the eyelid EL. The curvature 142, for example, enables the flexible support 44 to help force the heating strip 46 against the eyelid EL. Furthermore, the twist imparted along the flexible support may further help to transmit torsion along the length of the flexible support. In the absence of the curvature 142, as shown in FIG. 13B, the flexible support 44 may instead act as a spring which may pull the heating strip 46 away from the eyelid surface and reduce heat transmission during treatment.

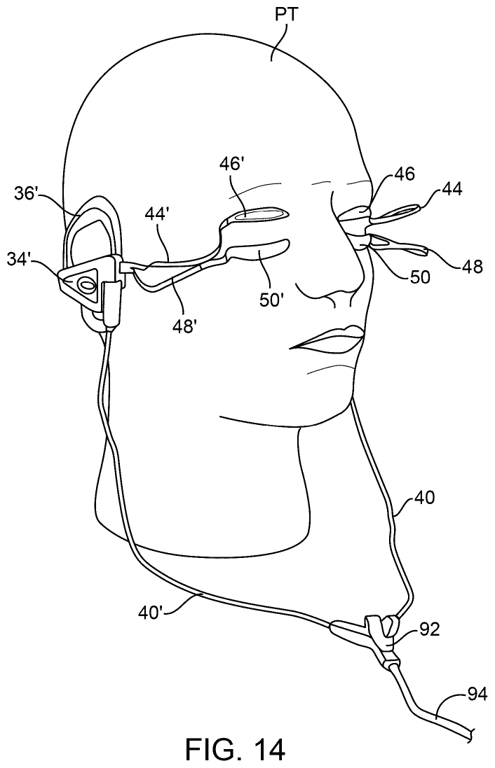

FIG. 14 shows an example of the lid treatment assemblies secured to each ear of the patient PT with each respective flexible support extending from the flex circuits. The treatment strips may be seen applied to each of the upper and lower lids of the patient PT and the cables 40, 40′ extending from each treatment assembly for coupling to a single yoke 92. The cable 94 extending from the yoke 92 may couple to the controller or hub 62.

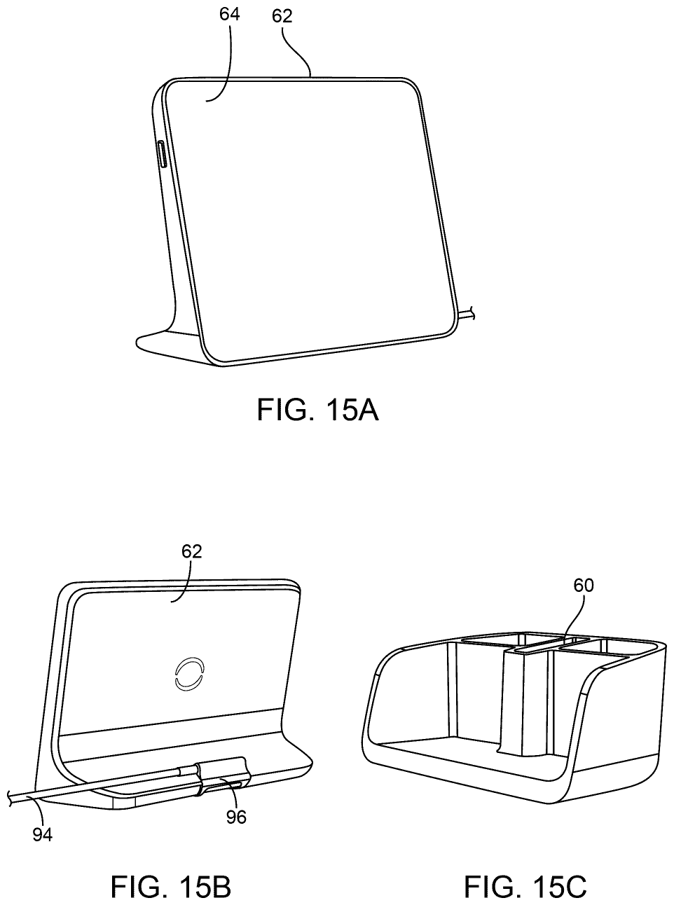

Turning now to the controller or hub 62, FIGS. 15A and 15B illustrate perspective front and back views of one variation of the controller or hub 62. FIG. 15B illustrates how the connector 96 which is in communication with the lid treatment assemblies may be electrically coupled to the controller or hub 62. The controller or hub 62 may be provided with the display 64 such as a touchscreen display which may re-orient the image upon the display 64 depending upon the orientation of the controller or hub 62. A front perspective view of the charging dock or nest 60 is illustrated in FIG. 15C for electrically coupling to and charging the controller or hub 62 and/or treatment assemblies.

The controller or hub 62 may provide for improved communication and interface with the practitioner and/or patient as the display 64 may provide for direct communication of therapy errors as well as providing a trouble shooting guide.

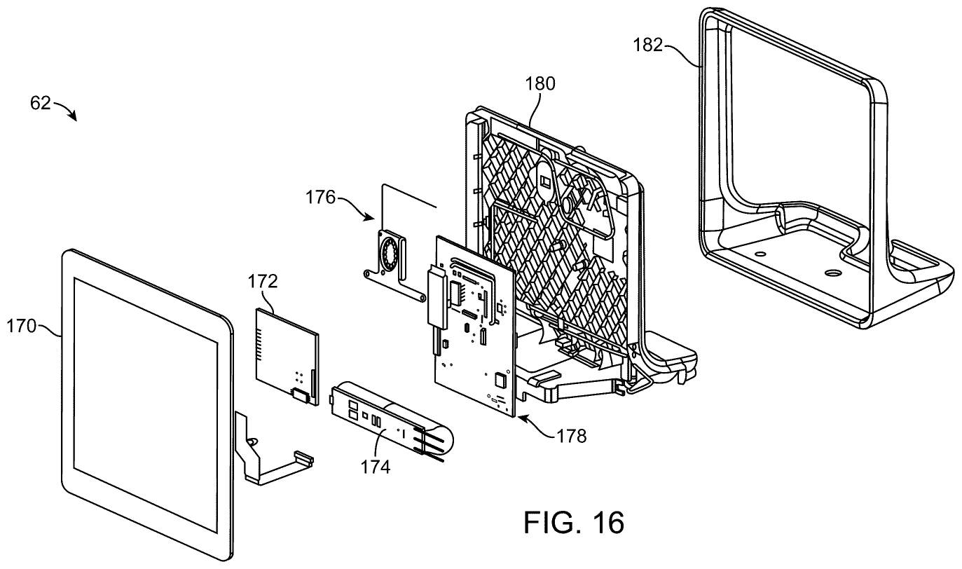

FIG. 16 shows an exploded perspective assembly view of one variation of the controller or hub 62 to illustrate how the controller may be configured. The display 170 may be front facing to the practitioner and/or patient and the back housing shell 180 and back housing frame 182 may enclose the electronics and various components within. A therapy control module controller or hub 172 may be provided along with a battery 174. The controller or hub 172 may include the processor and memory components which are programmed for applying the treatment protocols. A heat exchanger thermal management assembly 176 along with a main system interface PCBA 178 may also be provided for controlling the controlling or hub 62 display 170 and other components.

In one aspect, the controller or hub 62 may be programmed to provide a safety feature by monitoring the current applied to the heating strips. A parallel circuit within the controller or hub 62 may monitor the current of the heating strips in a closed loop feedback system. In the event that the current increases but no increase in temperature is sensed, e.g., by a thermistor or other temperature sensor on the heating strips, this may provide an indication to the controller or hub 62 that a heating strip is disconnected from the lid surface and that particular heating strip should be turned off. If the temperature increases but the current load remains unchanged, this may provide an indication to the controller or hub 62 that a disproportionate thermal load exists within the heating strip.

The controller or hub 172 may also be programmed to incorporate educational tools as well as a compilation of patient eye images for comparison and viewing purposes between visits, e.g., a miebography library of captured images of a patient over the course of several treatments. The treatment protocol provided by the controller or hub 172 may include temperature regulation protocols where the heating strips are continuously monitored by, e.g., a PID control system programmed to ensure that a maximum allowable temperature is not exceeded by the heating strips during treatment. The controller or hub 172 may also be programmed to provide an automatic temperature ramp, e.g., from 41° to 45° C. in five 1° C. steps increments. Furthermore, the controller or hub 172 may be further programmed to monitor a surface temperature of each heating element and initiate treatment therapy at a set point just above this sensed pre-heating surface temperature. The controller or hub 172 may increase the treatment temperature at a predetermined rate up to the system or practitioner-chosen maximum temperature setting. This may provide enhanced comfort for patients rather than initiating therapy at an initial setting that is pre-defined.

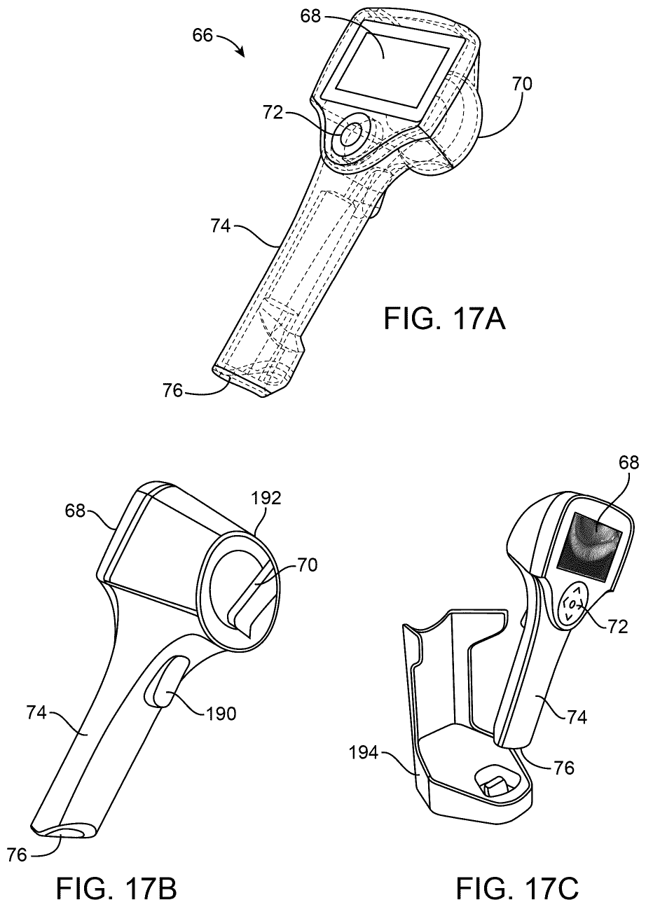

Turning now to the image capture device 66, perspective rear and front views of one variation of the image capture device are shown in FIGS. 17A and 17B. FIG. 17C illustrates a variation where the image capture device 66 may incorporate a stand 194 upon which the device 66 may be stored and/or charged. The device 66, which is a handheld and unsupported meibography camera which is portable, may incorporate an actuator 190 such as a trigger along the handle 74 for capturing an image and/or video of the eye. One or more lights 192 may also be incorporated to facilitate image capture.

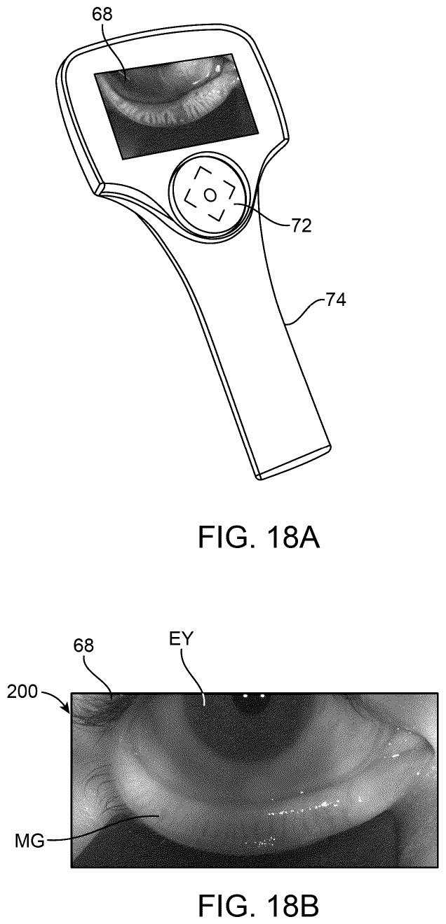

The image capture device 66 is further shown in FIG. 18A illustrating how a captured image 200, as shown in FIG. 18B, may be shown upon the display 68 of the device 66. The captured image 200 illustrates an example of the patient's eye EY and the meibomian glands MG to illustrate how various structures of the eye and eyelids may be captured and displayed for storage and/or analysis. The imager 70 may include, e.g., a HD infrared imager and, e.g., an LED array ring light 192 surrounding the imager 70. The control feature 72 may provide for various actions such as a menu control (e.g., zoom, accept, reject, etc.). The captured image or images may include, for example, unprocessed meibography images taken prior to, during, and/or after a treatment and also over one or more treatment sessions.

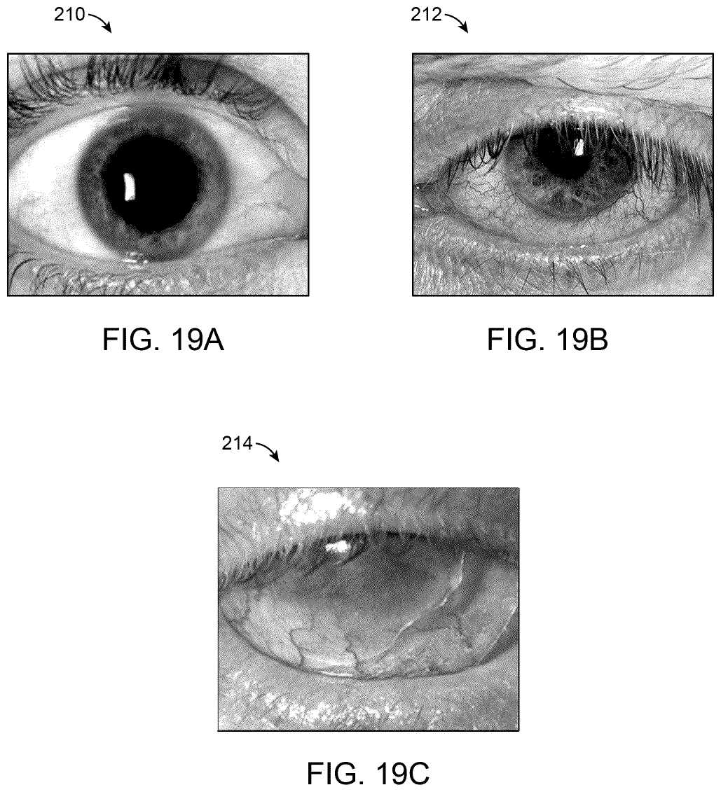

FIGS. 19A to 19C illustrate images 210, 212, 214 of various conditions of the patient's eye aside from the meibomian glands showing how the device 66 may be used to capture various eye conditions. The captured images may be transmitted to either the controller or hub 62 where the images may be processed locally and/or the images may be transmitted to a remote server where they may be processed remotely via artificial intelligence or machine learning processes to determine or characterize the condition or prognosis of the eye disorder.

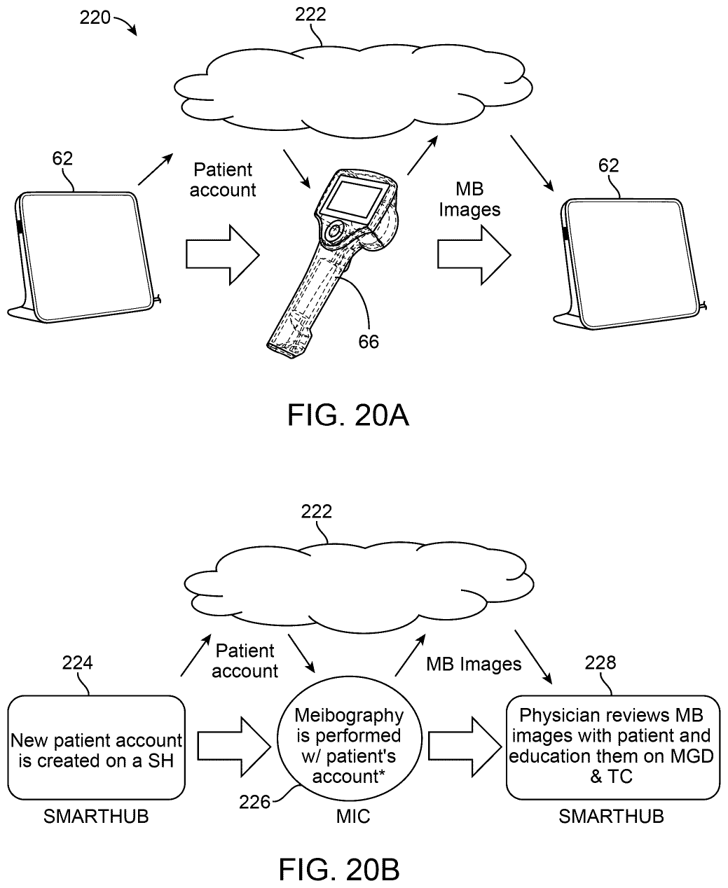

FIG. 20A illustrates one example for how a meibography system 220 may be implemented using the controller or hub 62 in combination with the image capture device 66. FIG. 20B illustrates another example functionally of how meibography may be implemented. Using the controller or hub 62, a new patient account may be created or accessed 224 where the controller or hub 62 may access the patient information from a remote cloud based server 222. The image capture device 66 may be used by the practitioner to capture one or more images of the eye and/or surrounding structures such as the meibomian glands under treatment 226. The one or more captured images may either be transmitted directly to the controller or hub 62 where image processing may be handled locally or the one or more captured images may be transmitted remotely to the cloud based server 222 where they may be processed remotely. The images may viewed directly upon the controller or hub 62 locally by the practitioner and/or patient and any processed information, for example, utilizing machine learning algorithms processed remotely by the server 222, may be transmitted to the controller or hub 62 where the practitioner may review the processed information for further treatment guidance.

The remote cloud based server 222 may be programmed to perform any number of functions such as identifying disease states of the eye and surrounding structures based upon the images received. In other alternative processes, the images may be processed and graded using an eye health criteria by assigning a quantitative score for assessment purposes. The health of the eye may be tracked over time by receiving and processing additional images received from each treatment session such that a graph or chart may be created tracking the patient's treatment progress over time.

Furthermore, the controller or hub 62 may be further programmed or configured to provide various additional functions or prompts, for example, blink prompting based on present or empirical datasets. The controller or hub 62 may also be programmed or configured to track and/or assess various eye-related conditions, for example, tear break up time, blink rate, tear meniscus height, tear volume, tear osmolarity, etc.

As described above, time-lapsed images from the patient may be obtained to display a progression of a disease or treatment progress. A grading system may be developed utilizing a predictive AI model based on the images and grading (e.g., on a scale of grade 0 to grade 3). The processing may occur either locally within the controller or hub 62 or via a cloud based server 222. The AI predictive algorithm may provide a prognosis of the disease progression to provide preventive care.

One example of a grading metric for grading meibomian gland disease may include the application of criteria. This may be particularly helpful as no standard metric is presently used for grading purposes aside from the assignment of mild, moderate, and severe for the meibomian gland disease. The images of the eye obtained by the device 66 may assigned a numerical value based a severity range from, e.g., 1 to 10, where 10 may be assigned to a worst-case severity. The assessment may be based upon criteria, e.g., such as the following:
Number of meibomian glands dropped out completely, e.g., not functioning at all or not functioning properly
Lengths of meibomian glands dropped out partially
Ratio of meibomian glands partially dropped out to existing meibomian glands
Location of meibomian gland dropped out for both partial and complete
Tortuosity of the glands as the more tortuous a gland, the more difficult for fluid to exit the gland
Thickness of glands as glands may be blocked in certain percentages, e.g., 10% blockage of a gland may play a role in determining whether patient will develop dry eye or not
Plumbness or volume of meibomian glands as the relative health of certain glands may determine whether a patient will develop dry eye or not; for example, a 70% loss for one patient may not develop dry eye but may for another patient
Tear break up time
Blink time and frequency

As images of the patient's eye are collected at every treatment, differences may be compared from each treatment session over time. At the time of image capture, various criteria may be assessed including, for example, meibographic images with assigned severity, tear break-up time scores with assigned severity, age, gender, race, etc. and the disease progression may be plotted over time. A severity grade based on the criteria described may be assessed at each time frame and the severity changes charted over time. Moreover, multiple patient data over time may be compiled to create a disease progression chart which may be customized for various criteria such as age, gender, race, or a combination thereof.

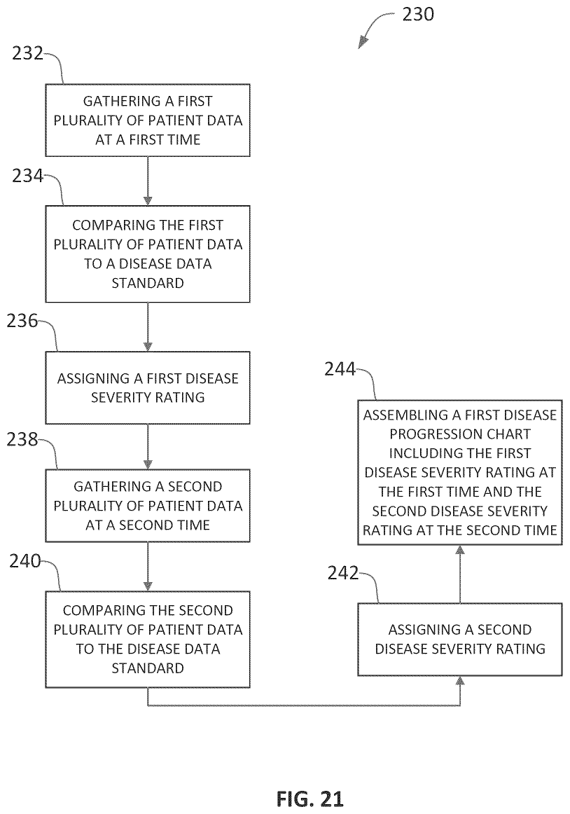

The flow diagram 230 of FIG. 21 illustrates how the grading system may be implemented in one example. A first plurality of patient data at a first time (e.g., first treatment or assessment session) may be gathered 232. The patient data may include the criteria and/or assessment described above. The plurality of patient data may be compared to a disease data standard 234 and a first disease severity rating may be assigned 236. As additional patient data is gathered and compiled a second plurality of patient data may be gathered 238 and may then be compared to the disease data standard 240 and a second disease severity rating may be assigned 242. Over time with sufficient data gathered, the disease progression chart including the patient data gathered over time (e.g., first disease severity rating, second disease severity rating, etc.) may be assembled 244.

The applications of the devices and methods discussed above are not limited to the treatment of dry eye syndrome but may include any number of further treatment applications. Moreover, such devices and methods may be applied to other treatment sites within the body where acute or chronic inflammation causes a disease or condition. The treatment strips can be accordingly custom-designed to follow the path of the underlying physiology, e.g. custom designed and contoured cooling or heating treatment strips to treat the sinuses and acute or chronic sinusitis, respectively, rhinitis and allergic rhinitis, joint aches and inflammation, arthritis, muscle aches, back pain, headaches, wounds, sports injuries, etc. Modification of the above-described assemblies and methods for carrying out the invention, combinations between different variations as practicable, and variations of aspects of the invention that are obvious to those of skill in the art are intended to be within the scope of the claims.

What is claimed is:

1. A system for treating eye-related dysfunctions, comprising:
a housing defining a receiving channel;
a first flexible support extending from the housing and having a first heating strip at a distal end of the first flexible support, wherein a proximal portion of the first flexible support defines a curved or bent portion which orients the first heating strip to extend in a first direction;
a second flexible support extending from the housing and having a second heating strip at a distal end of the second flexible support, wherein a proximal portion of the second flexible support defines a curved or bent portion which orients the second heating strip in a second direction,
wherein the first flexible support further defines a distal curved or bent portion proximal to the first heating strip and the second flexible support further defines a distal curved or bent portion proximal to the second heating strip such that the first heating strip and second heating strip are positioned to extend away from one another.

2. The system of claim 1 further comprising an ear attachment extending from the housing.

3. The system of claim 1 further comprising a flex circuit from which the first flexible support and the second flexible support each extend.

4. The system of claim 1 wherein the first heating strip further defines a first radius of curvature.

5. The system of claim 1 wherein the second heating strip further defines a second radius of curvature.

6. The system of claim 1 wherein the first heating strip is rotated in a direction opposite to the second heating strip.

7. The system of claim 1 further comprising a connector removably attachable to the receiving channel of the housing.

8. The system of claim 7 wherein the connector defines one or more orientation features for corresponding attachment to the receiving channel.

9. The system of claim 7 further comprising a magnetic feature contained within the housing for magnetic coupling to the connector.

10. The system of claim 1 further comprising a controller or hub in electrical communication through the housing.

11. The system of claim 10 wherein the controller or hub is programmed to provide a treatment therapy through the first heating strip and the second heating strip.

12. The system of claim 10 wherein the controller or hub is configured to communicate with a remote server.

13. The system of claim 12 wherein the remote server is configured to receive treatment data from the controller or hub, analyze the received treatment data, and return a customized treatment protocol based on the analyzed treatment data.

14. The system of claim 10 further comprising an image capture device configured to communicate with the controller or hub.

15. The system of claim 14 wherein the image capture device is configured to include an infrared imaging capability for capturing images under low light conditions.

16. The system of claim 14 wherein the image capture device is configured to capture one or more images of an eye or eye-related structure.

17. The system of claim 16 wherein the image capture device is further configured to analyze one or more captured images using machine learning algorithms to identify abnormalities in the eye.

18. The system of claim 10 further comprising one or more temperature sensors located adjacent to each of the first and second heating strips, wherein the one or more temperature sensors are configured to monitor a temperature of each of the first and second heating strips and provide feedback to the controller or hub.

19. The system of claim 18 wherein the controller or hub is programmed to adjust the temperature of each of the first and second heating strips based on feedback from the one or more temperature sensors to maintain a predetermined temperature range.

20. The system of claim 10 further comprising a user interface integrated into the housing or controller, wherein the user interface is configured to allow a user to select and customize one or more treatment modes and/or durations.

21. The system of claim 10 further comprising a memory within the controller or hub for storing one or more treatment histories and/or user profiles.

22. The system of claim 1 wherein each of the first and second heating strips comprises a layer of flexible, thermally conductive material configured to conform to contours of an eye area.

23. The system of claim 1 further comprising a power management system integrated within the housing, wherein the power manage system is configured to regulate power distribution to the first heating strip and the second heating strip.

24. The system of claim 1 further comprising a biocompatible material coated upon the first and second heating strips.
